(19) United States
(12) Patent Application Publication
(10) Pub. No.: US 2023/0302225 A1
(43) Pub. Date: Sep. 28, 2023

(54) PLUNGER AND FINGER GRIP FOR A SYRINGE ASSEMBLY, METHOD FOR MAKING THEM, SYRINGE ASSEMBLY, KIT AND USE OF THE SAME

(71) Applicant: MERZ PHARMA GMBH & CO. KGAA, Frankfurt am Main (DE)

(72) Inventors: Simon SCHMITT, Frankfurt am Main (DE); Uwe WORTMANN, Frankfurt am Main (DE); Dany BOROWEK, Frankfurt am Main (DE); Dirk NIPPRASCH, Frankfurt am Main (DE); Kai TIESBERGER, Bensheim (DE)

(21) Appl. No.: 18/043,663

(22) PCT Filed: Sep. 2, 2021
(86) PCT No.: PCT/EP2021/074265

§ 371 Date: Mar. 1, 2023

(30) Foreign Application Priority Data
Sep. 3, 2020 (EP) 20194478.2

(51) Int. Cl. A61M 5/31

(52) U.S. Cl. CPC A61M 5/3137

(57) ABSTRACT

The present invention relates to a plunger (20) and a finger grip (30) for a syringe assembly (100), methods for manufacturing them, to a syringe assembly (100), to a kit comprising parts of a syringe assembly (100), and to the use of a syringe assembly (100) or of a kit, wherein the syringe assembly (100) comprises a syringe barrel (10), a finger grip (30) having flanges (31, 32), and a plunger (20) having a plunger rod (21) and a plunger head (22). The plunger (20) comprises a grip pad (23) made by injection molding and attached to a proximal facing surface (24) of the plunger head. The finger grip (20) comprises a grip pad (35) also made by injection molding and attached to a distal facing surface of at least one flange (31, 32) of the finger grip (30).

### FIELD OF THE INVENTION

The present invention relates to a plunger for a syringe assembly for administering a liquid composition and to a method for manufacturing such a plunger, wherein the plunger comprises a plunger rod and a plunger head, wherein the plunger rod comprises a proximal end and a distal end, and wherein the plunger head is attached to the proximal end of the plunger rod and comprises a proximal facing surface configured to serve as a thumb plate during administering.

Further, the present invention relates to a finger grip for a syringe assembly for administering a liquid composition and to a method for manufacturing such a finger grip, wherein the finger grip comprises a finger grip body with two flanges extending radially in opposite directions from a center bore in the finger grip body through which a plunger of the syringe assembly can be inserted, each flange serving as a grip portion and having a finger contact area onto which at least one finger of a user can be put partly during administering to support the finger whilst administering, wherein each flange comprises a proximal facing surface and a distal facing surface.

Furthermore, the present invention relates to a syringe assembly for administering a liquid composition to a human or animal body, at least comprising a longitudinal syringe barrel having an inner lumen for receiving and storing a liquid composition to be administered, a finger grip coupled to proximal end of said barrel, and a plunger having a plunger rod and a plunger head, and the plunger rod being disposed at least partly within the inner lumen of the barrel with this barrel with its distal end.

Further the present invention relates to a kit comprising parts of a prescribed syringe assembly.

Moreover, the present invention relates to a use of a pre-described syringe assembly or pre-described kit for cosmetic or therapeutic application, particularly for replacing or filling a biological tissue or increasing volume of a biological tissue.

### BACKGROUND OF THE INVENTION

Syringe assemblies for administering a liquid composition and kits comprising several parts for assembling a syringe, in particular comprising at least a syringe barrel, a finger grip and a plunger having a plunger rod and a plunger head, are known from prior art for a broad range of applications.

From prior art an injection device is known, for example, including a syringe barrel having a body with a piston disposed therein with an open end is known, which can be pre-filled with a viscous fluid. A needle assembly is provided which includes a cannula having a locking connection engageable with the syringe distal end with a Luer connector including a hub. Mating engagement is provided by way of internal threads at the syringe distal end and external threads of said hub with a pitch sufficient to prevent detachment of the hub from the syringe distal end during ejection of the viscous fluid out of the syringe barrel. In addition, the specially designed hub further prevents detachment of the hub from the syringe distal end during ejection of the viscous fluid.

Further, a syringe assembly for delivering a fluid is known, wherein the syringe assembly comprises pusher means and a disposable cartridge configured to contain a fluid to be delivered, wherein the disposable cartridge comprises a container that defines a chamber for the fluid, and clothing means configured to hermetically insulate the chamber with respect to the outside.

Also, known from prior art is a syringe assembly for delivering a fluid, wherein the syringe assembly comprises pusher means and a disposable cartridge configured to contain a fluid to be delivered, wherein the disposable cartridge comprises a container that defines a chamber for the fluid and which is provided with a threaded collar to which fluid delivery means can be removably screwed.

Moreover, an aspiration and injection device is known that can allow economic, safe, and precise aspiration of the target site and ejection of a medicament to the target site. The device can include a syringe barrel, flange extender couplable to the barrel, and plunger that can be engaged by hand and/or one or more finger of the user to perform aspiration and injection at the target site.

Further, it is known to use a tip cap for closing and/or sealing the distal end of a syringe barrel.

Additionally, methods for manufacturing syringe assemblies and kits as prescribed are known in general, also for manufacturing of single parts for syringe assemblies or kits. In particular, methods for manufacturing syringe barrels, plungers and/or finger grips are known from prior art.

Further, a manufacturing method for a syringe barrel is known from prior art, including an administering step for applying a non-reactive silicone oil to the barrel inner surface by spraying the silicone oil thereto, and a heating step for heating the applied silicone oil to lower the viscosity thereof. The barrel inner surface of the syringe barrel is coated with the non-reactive silicone oil, which has a viscosity of 3000 to 30000 cps. The coating ratio of the silicone oil is no higher than 20%.

### OBJECTIVES OF THE INVENTION

In view of the above, one objective of the present invention is the provision of an alternative plunger, in particular of an improved plunger, for a syringe assembly for administering a liquid composition. Another objective is the provision of a method for manufacturing a plunger for a syringe assembly, the provision of an alternative finger grip for a syringe assembly, in particular of an improved finger grip, the provision of a method for manufacturing a finger grip, an alternative syringe assembly, in particular an improved syringe assembly, an alternative kit comprising parts of a syringe assembly, in particular an improved kit, and an alternative, in particular improved method for use of a syringe assembly or kit.

### SUMMARY OF THE INVENTION

The objects of the present invention are a plunger for a syringe assembly, a method for manufacturing such a plunger, a finger grip for a syringe assembly, a method for manufacturing such a finger grip, a syringe assembly, a kit comprising parts of the syringe assembly and a method of use of the syringe assembly or a kit each as defined according to the corresponding independent claim.

A first aspect of the present invention relates to a plunger for a syringe assembly for administering a liquid composition, wherein the plunger comprises a plunger rod and a plunger head. The plunger rod comprises a proximal end and a distal end, wherein the plunger head is attached to the proximal end of the plunger rod. The plunger head comprises a proximal facing surface configured to serve as a thumb plate during administering. According to the present invention the plunger comprises a grip pad attached to the proximal facing surface of the plunger head, wherein the grip pad extend at least partly over said proximal facing surface and is made by injection molding.

A second aspect of the present invention relates to a method for manufacturing a prescribed plunger, wherein the method comprises at least the steps:
manufacturing, in particular at least, the plunger head, and
forming the grip pad by injection molding.

A third aspect of the present invention relates to a finger grip for a syringe assembly for administering a liquid composition, wherein the finger grip comprises a finger grip body with two flanges extending radially in opposite directions from a center bore of the finger grip body, wherein through the center bore a plunger of a syringe assembly can be inserted. Each flange serves as a grip portion and has a finger contact area onto which at least one finger of a user can be put partly during administering to support the finger whilst administering. Further, each flange comprises a proximal facing surface and a distal facing surface. According to the present invention the finger grip comprises a grip pad attached to the distal facing surface of at least one of the flanges, wherein the grip pad extends at least partly over said distal facing surface and is made by injection molding.

A fourth aspect of the present invention relates to a method for manufacturing a prescribed finger grip, wherein the method comprises at least the steps:
manufacturing a finger grip body including two flanges extending radially in opposite directions from a center bore in the finger grip body, wherein a plunger of a syringe assembly can be inserted through said center bore, each flange serving as a grip portion and having a finger contact area onto which at least one finger of a user can be put partly during administering to support the finger whilst administering, wherein each flange comprises a proximal facing surface and a distal facing surface, and
forming the grip pad by injection molding.

A fifth aspect of the present invention relates to a syringe assembly for administering a liquid composition to a human or animal body, comprising at least a longitudinal syringe barrel having an inner lumen for receiving and storing a liquid composition to be administered, a finger grip coupled to a proximal end of said barrel, and a plunger having a plunger rod and a plunger head, the plunger rod being disposed at least partly within the inner lumen of the barrel with its distal end. According to the present invention the plunger is a plunger according to the present invention or a plunger made by a method according to the present invention, and/or the finger grip is a finger grip according to the present invention or a finger grip made by a method according to the present invention.

A six aspect of the present invention relates to a kit comprising several parts for a syringe assembly according to the present invention, wherein the parts are configured to be assembled to such a syringe assembly. The kit comprises at least a longitudinal syringe barrel having an inner lumen for receiving a liquid composition to be administered, a finger grip configured to be coupled to a proximal end of said barrel, and a plunger having a plunger rod and a plunger head, the plunger rod configured to be disposed at least partly within the inner lumen of the barrel with its distal end. According to the present invention the plunger is a plunger according to the present invention or a plunger made by a method according to the present invention, and/or the finger grip is a finger grip according to the present invention or a finger grip made by a method according to the present invention.

A seventh aspect of the present invention relates to a use of a syringe assembly according to the present invention or of a kit according to the present invention for cosmetic or therapeutic application, particularly for replacing or filling a biological tissue or increasing volume of a biological tissue, wherein an effective amount of a liquid composition, particularly an effective amount of a dermal filler composition, is administered to a subject.

The present invention allows providing of a very easy, comfortable and safe to use syringe assembly for administering of a liquid composition, in particular for injection of dermal filler, in particular for administering of hyaluronic acid dermal filler, respectively of a very easy, comfortable and safe to use kit comprising parts for a syringe assembly, wherein the syringe assembly and/or the kit respectively the single parts of said kit and/or the syringe assembly are easy to manufacture and enable good storage and an easy handling by a practitioner.

By the grip pad attached to the proximal facing surface of the plunger head and/or by the grip pad attached to the distal facing surface of one of the flanges of the finger grip body, a plunger with a plunger head with reduced slippery and a comfortably finger contact area respectively a finger grip with reduced slippery and a comfortably finger contact area can be provided. Thereby, a more comfortable, precise and safer to use syringe assembly or kit can be provided. The reduced finger slippage results in a more controlled use, in particular in case of injections of high viscous fluids, as for example in case of injection or administering of dermal filler, in particular of hyaluronic acid dermal filler, which require high extrusion forces.

Possible and in particular preferred embodiments of the objects of the present invention are set forth in the dependent claims. Further embodiments and other objects, advantages and features of the present invention will become apparent from the following detailed description of the invention, the illustration of particular embodiments in the accompanying figures and the detailed description of the embodiments. By express reference, the wording of the original claims is herewith incorporated to the description.

### DETAILED DESCRIPTION OF THE INVENTION

A plunger for a syringe assembly for administering a liquid composition according to the first aspect of the present invention comprises a plunger rod and a plunger head, wherein the plunger rod comprises a proximal end and a distal end, and wherein the plunger head is attached to the proximal end of the plunger rod and comprises a proximal facing surface configured to serve as a thumb plate during administering. According to the present invention the plunger further comprises a grip pad attached to the proximal facing surface of the plunger head, wherein the grip pad extends at least partly over said proximal facing surface and is made by injection molding.

By the grip pad attached to the proximal facing surface of the plunger head a plunger head with a comfortably finger contact area with reduced slippery can be provided. Thereby, a more comfortable, precise and safer to use syringe assembly comprising a plunger can be provided. The reduced finger slippage achieved by the grip pad greatly improves safety during use, in particular during injections. By the grip pad a better grip can be achieved, what results in a more controlled use, in particular when the plunger is used in a syringe assembly for injection of high viscous fluids, as for example for injection or administering of dermal filler, in particular for hyaluronic acid dermal filler, which require high extrusion forces. Using injection molding for manufacturing of the grip pad for the plunger head enables very ease and economic manufacturing of the grip pad.

The term “syringe assembly” as used herein in general refers to an application device, in particular to an injection device, being configured for discharging, preferably administering, in particular for injecting, of at least one component received and stored in that device. The term “syringe assembly” in particular encompasses application devices, in particular injection devices, for discharging one-, two- or multi-component content, in particular devices for discharging one-, two- or multi-component materials. The term “syringe assembly” as used herein in particular refers to a syringe or a syringe-like application device being configured for discharging or administering at least one component stored in that device. The term “syringe assembly” as used herein further in particular refers to a device operating as a manually operating reciprocating pump, usually comprising a syringe barrel having an inner lumen or chamber for receiving and storing at least one component to be administered, and a plunger, wherein the plunger in particular can be pulled and pushed along the inside of the inner lumen or chamber along a longitudinal axis of the inner lumen, allowing to take in and expel in particular a fluid through a discharge orifice at a proximal end of the inner lumen or chamber respectively at the proximal end of the syringe barrel.

Within the context of the use of a syringe assembly and/or a kit according to the present invention, the term “injection” may refer to intra-, inter- or subdermal injection or subcutaneous injection.

The term “composition”, as used herein, is not particularly limited and may be, e.g., a liquid composition. Preferably, the composition may be a composition that is used or suitable for use as a soft tissue filler, in particular a dermal filler.

The term “liquid composition”, as used herein, should be understood to encompass a liquid (e.g., an aqueous liquid), a solution (e.g., an aqueous solution), a suspension (e.g., an aqueous suspension), a dispersion (e.g., an aqueous dispersion), an emulsion (e.g., an aqueous emulsion), a gel (e.g., a hydrogel) etc., and mixtures thereof.

The term “multi-component content”, as used herein, refers to a content or composition comprising at least two (different) components, in particular more than two (different) components, which have to mixed to one mixture before administering. Accordingly, a one-component composition is a composition being ready to use and a two-component composition a composition having two components which have to be mixed before administering.

The term “plunger” as used herein refers to a part made in one piece or to an assembly of several parts, configured to be used in a syringe assembly for applying an extrusion force to a content, for example a liquid composition, received and stored in an inner lumen or chamber of a syringe barrel, wherein by the plunger the content can be pressed out of the inner lumen of the syringe barrel, when a sufficient extrusion force is applied in longitudinal direction causing longitudinal movement of the plunger within the inner lumen of the syringe barrel along the longitudinal axis of the syringe barrel.

The term “plunger rod” or “push rod” as used herein refers in particular to a longitudinal, preferably bar-like part configured to be inserted at least partly with its distal end in an inner lumen or chamber of a syringe barrel.

The term “plunger head” as used herein refers to a part attached to the proximal end of the plunger rod, serving as a thumb plate during administering. For this purpose, in particular the plunger head comprises a substantially flat proximal facing surface, which is preferably configured for supporting a thumb of a medical practitioner at use, in particular during applying an extrusion force required for dispensing a content received and stored in an inner lumen or chamber of a syringe barrel.

In a possible, in particular preferred embodiment of a plunger according to the present invention, the plunger head may comprise or may be a part that is shaped substantially as a plate or a substantially flat disc.

In a possible, in particular preferred embodiment of a plunger according to the present invention, the plunger head may be integrally manufactured with the plunger rod, in particular also by injection molding.

In an alternative embodiment of a plunger according to the present invention, the plunger head may be connected to the plunger rod, for example, by a snap-fit connection, in particular by a detachable snap-fit connection or by another mounting mechanism.

In a possible, in particular preferred embodiment of a plunger according to the present invention, the plunger may be configured for use in a syringe assembly comprising a syringe barrel configured for receiving and storing an amount of 2 ml or less of a composition to be administered, wherein the plunger may in particular be configured for use with a syringe barrel configured for receiving and storing an amount of 1 ml or less, for example of 0.5 ml, of a composition, in particular of a liquid composition of a dermal filler, as for example of a hyaluronic acid dermal filler.

In a possible, in particular preferred embodiment of a plunger according to the present invention, the plunger may be, in particular additionally, configured to be used for dispensing or extruding a composition received and stored in an inner lumen of a syringe barrel out of said inner lumen, wherein said inner lumen of said syringe barrel is defined by a cylindrical chamber having a smaller inner diameter and/or a larger dimension in longitudinal direction compared to the dimensions of an inner lumen of a 1 ml-syringe barrel according to standard ISO 11040-6.

In a possible, in particular preferred embodiment of a plunger according to the present invention, the grip pad on the plunger head is made of thermoplastic elastomere (TPE), in particular of a coloured thermoplastic elastomere (cTPE). With an injection moulded TPE grip pad a more comfortable, precise and safe usage and preventing of slipping of a user's thumb during use can be achieved. TPE as grip pad material is easy to apply on the plunger head proximal facing surface and a good adhesion to the material of the plunger head proximal facing surface can be achieved.

To achieve an advantageous result for providing an advantageous plunger, the plunger head can be made of polypropylene (PP), in particular also by injection molding. Preferably, the plunger rod is made of polypropylene, too, in particular also by injection molding, preferably in one piece with the plunger head. That means, in one embodiment of a plunger according to the present invention, the plunger is made by 2K-injection molding, wherein preferably the plunger rod and the plunger head each are made of the same first injection molding material, preferably in one piece, in particular of polypropylene (PP), wherein the grip pad is in particular made of a second material by injection molding, in particular of TPE.

Preferably, the grip pad is applied directly onto the proximal facing surface of the plunger head, when it is made. Alternatively, that means in another embodiment, the grip pad can also be made separately from the plunger respectively the plunger head and later be attached to the proximal facing surface of the plunger head.

Also, in another embodiment of a plunger according to the present invention, plunger rod and/or plunger head may be made of another material than PP, in particular of another suitable plastic. However, the material the plunger is made of, in particular has similar properties to PP.

Instead of TPE, the grip pad can also be made of another material, in particular of another suitable plastic. However, the chosen grip pad material is preferably a rubber-like material or another high friction generating material, which also prevents slipping of a user's thumb and provides a comfortable, precise and safe usage.

Making the plunger by 2K-injection molding allows enabling a good adhesion between the grip pad on the plunger head proximal facing surface and easy and advantageous manufacturing of the plunger.

In one embodiment according to the present invention, the grip pad may at least partly be coloured, in particular completely, wherein the grip pad can be made of a coloured material, for example and as already mentioned above, of coloured TPE. This can for example be achieved by adding colour particles to the injection molding raw material. The use of a plunger having a coloured grip pad allows providing of several syringe assemblies or kits, each prefilled with a (different) composition for administering, wherein each composition or content, is preferably associated to a defined colour by which the syringe assemblies and/or kits can be distinguished. This helps to differ between different products respectively different syringe assemblies or kits prefilled with different compositions for different use cases by a medical practitioner at a first glance. Thereby, the coloured grip pad helps greatly to improve usability and safety. In particular, the risk of mixing up syringe assemblies or kits can be achieved, since the coloured grip pad can be detected much more easily optically than any hint or any indication in attached instructions of use. Hence, by a coloured grip pad the risk of mis-administering, in particular of mixing up the compositions to be administered, can be reduced in a comfortable and easy way.

For good adhesion of the grip pad on the proximal facing surface of the plunger head, in one embodiment of the present invention, the plunger head can be made by using an injection molding tool having a surface by which the proximal facing surface of the plunger head is shaped and which has been eroded resulting in a tool surface roughness in a range from VDI 3400 Ref. 21 to VDI 3400 Ref. 30, i.e. in a roughness in between a range of Ra=1.12 μm to Ra=3.15 μm with Rmax in a range from 4.5 μm to 12.5 μm, wherein in particular the tool surface has been eroded such that the surface roughness results in a roughness according to VDI 3400 Ref. 27, characterised by Ra=2.25 μm and Rmax=9.0 μm.

To further improve the adhesion of the grip pad onto the proximal facing surface of the plunger head, in one embodiment of a plunger according to the present invention the proximal facing surface of the plunger head may comprise at least one deepening, in particular at least one groove, wherein, when the grip pad is attached to the proximal facing surface of the plunger head, said at least one deepening is filled by grip pad injection molding material. By such a deepening a larger contact zone area between the proximal facing surface and the grip pad material can be achieved what results in better adhesion respectively connection between the plunger head material and the grip pad material.

Preferably, the depth in a direction perpendicular to the proximal facing surface of at least one deepening is between 0.5 mm and 2 mm, preferably roundabout 1 mm.

In a very advantages embodiment of a plunger according to the present invention, the proximal facing surface of the plunger head may comprise two of such deepenings or grooves, in particular circular grooves being arranged concentric, preferably with their center aligned to the longitudinal axis of the plunger.

For comfortable and safe grip, in particular for comfortable and safe contact of a thumb of a user, the average thickness of the grip pad, in particular in the area outside of the grooves and/or deepening, is preferably in a range from 0.1 mm to 0.5 mm, in particular round about 0.2 mm.

In one embodiment of a plunger according to the present invention, the grip pad on the plunger head is designed in such a way that it represents a symbol, in particular a letter or another character, preferably selected from the ASCII-code. Thereby, particularly in addition or alternatively to a coloured grip pad, a plunger having an optically detectable distinguishing feature can be provided in a very easy way. This helps to differ between different products respectively different syringe assemblies or kits prefilled with different compositions for different use cases by a medical practitioner at a first glance. This helps greatly to improve usability and safety. In particular, the risk of mixing up syringe assemblies or kits can be achieved, since the symbol in the grip pad can be detected much more easily optically than any hint or indication in attached instructions of use. Hence, by a such a grip pad the risk of mis-administering, in particular of mixing up the composition's to be administered, can be reduced in a comfortable and easy way.

For example, the grip pad may be designed such that it represents one of the following symbols: “+”, “−”, “L” or similar. “+” or “L” for example may indicate, that a composition to be administered and being received and stored in an inner lumen of a syringe barrel of a syringe assembly or a kit contains a defined active ingredient, for example an anaesthetic as Lidocaine or the like. “−” for example may indicate that the composition a syringe assembly or a kit includes does not contain that or a defined active ingredient.

In one embodiment of a plunger according to the present invention, the symbol may be represented by areas not being filled or covered with grip pad injection molding material but are at least partially surrounded by grip pad injection molding material. That means, the symbol may be visualised by negative representation.

Alternatively, but also possible and in some cases more advantageous, the symbol may be represented by areas being filled or covered with grip pad injection molding material which are at least partially or completely surrounded by void areas. That means, in this case the symbol is visualised by positive representation.

In a possible, in particular preferred embodiment of a plunger according to the present invention, the grip pad attached to the plunger head is made in one piece by injection molding, in particular comprising one injection point only. Thereby, very easy manufacturing with only one injection molding step for generating the grip pad can be achieved. This allows a fast, easy and reproduceable manufacturing of the grip pad.

In one embodiment of a plunger according to the present invention, the plunger comprises only one grip pad, which is preferably made in one piece by injection molding, wherein in particular for forming the grip pad, the grip pad injection molding material has been applied directly onto the proximal facing surface of the plunger head. Thereby, very easy manufacturing with only one injection molding step for generating the grip pad can be achieved. This allows a fast and easy to reproduce manufacturing of the grip pad.

In one embodiment of a plunger according to the present invention, at least one injection point for the grip pad material is located near an outer circumference of the grip pad, in particular in an outer third of a diameter of the grip pad or in an outer third of a distance from the center of the grip pad to the outer circumference of the grip pad, in particular if the grip pad is made in one piece comprising one injection point only. This allows a very homogenous injection molding material distribution, in particular in case the proximal facing surface of the plunger head has a slightly concave shape to enable a more economic contact area for a user's thumb.

In one embodiment of the plunger according to the present invention, at its distal end the plunger rod may further be configured to receive a piston, wherein the plunger rod in particular may be configured to be connected to the piston with its distal end by a snap-fit connection. Thereby, a plunger can be provided, to which a piston can easily and space-savingly be mounted. A space-saving design is particularly necessary for a use of the plunger with a syringe barrel having a small inner diameter.

To achieve an advantageous snap-fit connection between the distal end of the plunger rod and the piston, in particular the piston's proximal end, the plunger rod may have a frustoconical-shaped tip or a mushroom-shaped tip or the like. Further, the plunger rod may have a piston receiving portion arranged adjacent to the tip in proximal direction with a smaller diameter than the tip. For an advantageous snap-fit connection, the piston may be made of or may contain at least partly a suitable, biocompatible elastomeric material.

With the frustoconical or mushroom shaped plunger rod tip geometry at the distal end of the plunger rod an almost errorless assembly of a piston to the distal end of the plunger rod can be achieved without the need to exert a torque on the plunger rod while a firm and secure connection can be established. Further this geometry is easier to manufacture and is space-saving compared to threaded connection between the distal end of the plunger rod and a piston.

For axially fixation of the piston at the distal end of the plunger rod and transfer of applied extrusion force, at the proximal end of the piston receiving portion the plunger rod may comprise a radial extending plate disc serving as a stop in proximal direction.

In one embodiment of a plunger according to the present invention, the plunger, in particular the plunger rod, may be configured to be subjected with high extrusion forces as they appear, for example, during ejection of high viscous fluids, as for example during ejection of dermal filler like hyaluronic acid dermal fillers or the like.

To achieve a sufficient mechanical strength of the plunger, in particular of the plunger rod, in particular to achieve a sufficient kink stability, that means sufficient resistance against kinking, in particular if the plunger is configured to be used with a 1 ml syringe barrel according to standard ISO11040-6 or a with syringe barrel having smaller inner diameter as such a 1 ml standard syringe barrel, the plunger rod may comprise at least one section along its longitudinal direction having a at least substantially double-T-profile shape, a double-T-profile-like shape, an I-profile shape or an I-profile-like shape or similar. For improved mechanical strength the plunger rod may have at least one section in its longitudinal direction having a double-mushroom-shaped-profile or a mushroom-shape-profile-like or a combined mushroom-T-shaped-profile or the like, in particular with a convex or curved outer geometry. In several cases, it would be advantageous, if the plunger rod is shaped like prescribed over its complete length. A good mechanical strength of the plunger, in particular of the plunger rod, enables the provision of a plunger being suitable for ejection and administering of high viscous fluids as for example of dermal fillers like hyaluronic acid dermal fillers. With a higher mechanical strength the probability of mechanical failure can be reduced and thereby safety can be increased.

For achieving a high kink stability respectively a high mechanical strength of the plunger rod, in some cases, a massive cross-section of the plunger rod may be more advantageous, in particular an oval or a circular or a square cross-section, wherein said massive, in particular oval or circular or square cross-section, may extend only over one section of the length of the plunger rod or over several sections in longitudinal direction of the plunger rod or may extend over the complete length of the plunger rod. The geometry and/or size of the cross-section area of the plunger rod, in particular the diameter, may be constant or varying over the length of the plunger rod. However, a constant geometry and/or size of the cross-section area is preferred in longitudinal direction of the plunger rod within at least one section of the plunger rod. The geometry and/or size of the cross-section area, in particular the diameter of the cross-section area of the plunger rod, may differ between several sections of the plunger rod or may be the same over the whole length of the plunger rod. In one embodiment, the geometry and/or size of the cross-section area of the plunger rod may be constant over at least 50%, 60%, 70%, 80%, 90% or 95% of the complete length of the plunger rod. Thereby, in some cases, advantageous mechanical properties can be achieved. This further enables easier manufacturing of the plunger rod. In some cases, it also enables the use of another, in particular more advantageous, push rod material in some cases.

The second aspect of the present invention is a method for manufacturing a plunger as pre-described, wherein according to the present invention a method for manufacturing such a plunger comprises at least the steps: manufacturing, in particular at least, of the plunger head, and forming the grip pad by injection molding.

In one embodiment of a method for manufacturing a plunger according to the present invention, the plunger head may also be made by injection molding, in particular in one piece with the plunger rod, wherein the plunger head and/or the plunger rod preferably are made of polypropylene (PP), in particular formed integrally in one piece.

Alternatively, plunger head and/or plunger rod may be made separately and/or of different material, wherein plunger rod and plunger head in particular each are made of a material having similar properties to PP.

In one embodiment, in particular in a preferred embodiment of a method for manufacturing a plunger according to the present invention, the grip pad is formed by applying the grip pad injection molding material directly onto the proximal facing surface of the plunger head, wherein the grip pad is particular made in one piece, preferably with just one injection point, which is in particular located in an outer third of the grip pad in radial direction.

A third aspect of the present invention relates to a finger grip for a syringe assembly for administering a liquid composition, wherein the finger grip according to the present invention comprises a finger grip body with two flanges or wings extending radially in opposite directions from a center bore in the finger grip body, wherein through said center bore a plunger of a syringe assembly, in particular a plunger according to the present invention, can be inserted. Each flange is configured to serve as a grip portion and has a finger contact area onto which at least one finger of a user can be put partly during administering to support the finger whilst administering, wherein each flange comprises a proximal facing surface and a distal facing surface. According to the present invention, the finger grip comprises a grip pad attached to the distal facing surface of at least one of the flanges or wings of the finger grip body, wherein the grip pad is extending at least partly over said distal facing surface and is made by injection molding.

By the grip pad attached to the distal facing surface of the finger grip a finger grip with a comfortably finger contact area with reduced slippery can be provided. Thereby, a more comfortable, precise and safer to use syringe assembly comprising a finger grip can be provided. The reduced finger slippage achieved by the grip pad greatly improves safety during use, in particular during injections. By the grip pad a better grip can be achieved, what results in a more controlled use, in particular when the finger grip is used in a syringe assembly for injection of high viscous fluids, as for example for injection or administering of dermal filler, in particular for hyaluronic acid dermal filler, which require high extrusion forces. Using injection molding for manufacturing of the grip pad for the finger grip enables very ease and economic manufacturing of the grip pad.

The term “finger grip” as used herein refers to a handle respectively a grasping device configured to be grasped at least partly by fingers of a user, in particular of a medical practitioner, for supporting at least a first finger and a second finger during administering while the finger grip is used in a syringe assembly.

The finger grip is in particular configured to be coupled, preferably detachably, to a syringe barrel, in particular to a proximal end of a syringe barrel, to enable a comfortable grasping of the syringe barrel for administering a composition received and stored within an inner lumen of said syringe barrel.

For easy coupling to a syringe barrel the finger grip, in particular the finger grip body, may comprise a slot into which a proximal flange of a syringe barrel can be inserted laterally as disclosed for example in WO 2017/176476 A1 or similar.

The center bore of the finger grip body may be partly open along its circumference or completely closed in circumferential direction. A center bore designed open in circumferential direction allows pre-assembling of syringe barrel and plunger, i.e. inserting of the plunger at least partly into the inner lumen of said barrel, before coupling of finger grip body and syringe barrel and inserting also the plunger laterally into said center bore. A center bore having a completely closed circumference requires insertion of the plunger after coupling of syringe barrel and finger grip body, wherein the plunger has to be inserted along the longitudinal axis of the syringe barrel and perpendicular to a center bore diameter.

Preferably the finger grip, in particular the two flanges of the finger grip, is/are configured to be grasped with one finger each. For ergonomic grasping of the finger grip, in particular at least one of the flanges is curved, preferably having a concave proximal facing side and a convex distal facing side. Preferably, one of the flanges or wings is configured to be grasped with the index finger, and the other one with the middle finger of a user's hand.

In a possible, in particular preferred embodiment of a finger grip according to the present invention, the finger grip may be configured for use in a syringe assembly comprising a syringe barrel configured for receiving and storing an amount of 2 ml or less of a composition to be administered, wherein the finger grip may in particular be configured for use, in particular coupling, with a syringe barrel configured for receiving and storing an amount of 1 ml or less, for example of 0.5 ml, of a composition, in particular of a liquid composition of a dermal filler, as for example of a hyaluronic acid dermal filler.

In one embodiment of a finger grip according to the present invention, at least one grip pad of the finger grip is made by applying the grip pad injection molding material directly onto the distal facing surface of the corresponding flange. Alternatively, that means in another embodiment, the grip pad can also be made separately from the finger grip respectively the finger grip body and later be attached to the distal facing surface of the corresponding flange or wing of the finger grip.

In one embodiment of the finger grip according to the present invention, at least one grip pad of the finger grip is made of thermoplastic elastomere (TPE), in particular of a coloured thermoplastic elastomere (cTPE).

With an injection moulded TPE grip pad a more comfortable, precise and safe usage and preventing of slipping of user's fingers during use can be achieved. TPE as grip pad material is easy to apply on the finger grip flanges' distal facing surfaces and a good adhesion to the material of the flange surfaces can be achieved.

To achieve advantageous result or for providing an advantageous finger grip, the finger grip body can be made of polypropylene (PP), in particular also by injection molding. That means, in one embodiment of a finger grip according to the present invention, the finger grip is made by 2K-injection molding, wherein preferably the finger grip body including the flanges is made of a first injection molding material, preferably in one piece, in particular of polypropylene (PP), and the grip pad is made of a second material by injection molding, in particular of TPE.

In another embodiment of a finger grip according to the present invention, the finger grip, in particular the finger grip body, may be made of another material than PP, in particular of another suitable plastic material. However, the material the finger grip is made of, in particular has similar properties to PP.

Instead of TPE, the grip pad can also be made of another material, in particular of another suitable plastic material. However the chosen grip pad material is preferably a rubber-like material or another high friction generating material, which also prevents slipping of user's fingers and provides a comfortable, precise and safe usage.

Making the finger grip by 2K-injection molding allows enabling a good adhesion between the grip pad on at least one of the distal facing surfaces of the finger grip body and an easy and advantageous manufacturing of the finger grip.

In one embodiment according to the present invention, the grip pad attached to the finger grip body may at least partly be coloured, in particular completely, wherein the grip pad can be made of a coloured material, for example and as already mentioned above, of coloured TPE. This can for example be achieved by adding colour particles to the injection molding raw material. The use of a finger grip having a coloured grip pad allows providing of several syringe assemblies or kits, each prefilled with a (different) composition for administering, wherein to each composition or content, preferably a defined colour is associated by which the syringe assemblies and/or kits can be distinguished. This helps to differ between different products respectively different syringe assemblies or kits prefilled with different composition's for different use cases by a medical practitioner at a first glance. Thereby, the coloured grip pad helps greatly to improve usability and safety. In particular, the risk of mixing up syringe assemblies or kits can be achieved, since the coloured grip pad can be detected much more easily optically than any hint or indication in attached instructions of use. Hence, by a coloured grip pad the risk of mis-administering, in particular of mixing up the composition's to be administered, can be reduced in a comfortable and easy way.

For good adhesion of the grip pad on at least one of the distal facing surfaces of the finger grip body in one embodiment of the present invention the finger grip body can be made by using an injection molding tool having a surface by which the distal facing surface of the finger grip body is shaped, wherein said tool surface has been eroded resulting in a tool surface roughness in a range from VDI 3400 Ref. 21 to VDI 3400 Ref. 30, i.e. resulting in a roughness in between Ra=1.12 μm to Ra=3.15 μm with Rmax in a range from 4.5 μm to 12.5 μm, wherein in particular the tool surface has been eroded such that the surface roughness results in a roughness according to VDI 3400 Ref. 27, characterised by Ra=2.25 μm and Rmax=9.0 μm.

To further improve the adhesion of the grip pad onto at least one of the distal facing surfaces of the finger grip body, in one embodiment of a finger grip according to the present invention at least one of the distal facing surfaces of the finger grip body may comprise at least one deepening, in particular at least one groove, wherein, when the grip pad is attached to the said surface of the finger grip body, said at least one deepening is filled by grip pad injection molding material. By such a deepening a larger contact zone area between the distal facing surface and the grip pad material can be achieved what results in better adhesion respectively connection between the finger grip body material and the grip pad material.

Preferably, the depth in a direction perpendicular to the distal facing surface of at least one deepening is between 0.5 mm and 2 mm, preferably roundabout 1 mm.

For comfortable and safe grip, in particular for comfortable and safe contact of fingers of a user, the average thickness of at least one grip pad of the finger grip, in particular in the area outside of the grooves and/or deepening, is preferably in a range from 0.1 mm to 0.5 mm, in particular round about 0.2 mm.

In one embodiment of a finger grip according to the present invention, the grip pad on the finger grip is designed in such a way that at least one area of a distal facing surface of the finger grip not being filled or covered with grip pad injection molding material is completely surrounded by injection molding material.

In a preferred embodiment of a finger grip according to the present invention, the area not being filled or covered with grip pad injection molding material comprises one or more stripes, in particular at least two parallel stripes, preferably extending in longitudinal direction of the corresponding flange or wing respectively in transversal direction of the finger grip.

The grip pad on the finger grip can alternatively be designed in such a way that it represents a symbol, in particular a letter or another character, preferably selected from the ASCII-code. Thereby, particularly in addition or alternatively to a coloured grip pad, a finger grip having an optically detectable distinguishing feature can be provided in a very easy way. This helps to differ between different products respectively different syringe assemblies or kits prefilled with different compositions for different use cases by a medical practitioner at a first glance. This helps greatly to improve usability and safety. In particular, the risk of mixing up syringe assemblies or kits can be achieved, since the symbol in the grip pad can be detected much more easily optically than any hint or indication in attached instructions of use. Hence, by a such a grip pad the risk of mis-administering, in particular of mixing up the composition's to be administered, can be reduced in a comfortable and easy way.

Also the grip pad on the finger grip may be designed such that it represents one of the following symbols: “+”, “−”, “L” or similar.

In one embodiment of a finger grip according to the present invention, the symbol may be represented by areas not being filled or covered with grip pad injection molding material which are at least partially surrounded by grip pad injection molding material. That means, the symbol may be visualised by negative representation.

Alternatively, but also possible and in some cases more advantageous, the symbol may be represented by areas being filled with grip pad injection molding material which are at least partially or completely surrounded by void areas. That means, in this case the symbol is visualised by positive representation.

In a possible, in particular preferred embodiment of a finger grip according to the present invention, the grip pad attached to the finger grip body is made in one piece by injection molding, in particular comprising one injection point only. Thereby, very easy manufacturing with only one injection molding step for generating the grip pad can be achieved. This allows a fast and easy to reproduce manufacturing of the grip pad.

In one embodiment of a finger grip according to the present invention, the each flange of the finger grip body comprises only one grip pad, which is preferably made in one piece by injection molding, wherein in particular for forming the grip pad, the grip pad injection molding material has been applied directly onto the distal facing surface of said flange. Thereby, very easy manufacturing with only one injection molding step for generating the grip pad can be achieved. This allows a fast and easy to reproduce manufacturing of the grip pad.

In one embodiment of a plunger according to the present invention, at least one injection point for the grip pad material is located near the outer circumference of the grip pad, in particular in an outer third of a diameter of the grip pad or in an outer third of a distance from the center of the grip pad to the outer circumference of the grip pad, in particular if the grip pad is made in one piece comprising one injection point only. This allows a very homogenous injection molding material distribution.

The fourth aspect of the present invention relates to a method for manufacturing a finger grip as pre-described, wherein according to the present invention a method for manufacturing such a finger grip comprises at least the steps:
manufacturing a finger grip body including two flanges or wings extending radially in opposite directions from a center bore in the finger grip body, wherein a plunger of the syringe assembly can be inserted through this center bore, and wherein each flange serves as a grip portion and has a finger contact area onto which at least one finger of a user can be put partly during administering to support the finger whilst administering, and wherein each flange comprises a proximal facing surface and a distal facing surface, and
forming the grip pad by injection molding.

In one embodiment, in particular in a preferred embodiment, the finger grip is also made from plastic, in particular from polypropylene, in particular also by injection molding. Preferably, the finger grip is completely made by 2K-injection molding, wherein the finger grip body is preferably made of a first injection molding material and the grip pad is made of a second injection molding material, in particular of TPE.

In one embodiment, in particular in a preferred embodiment of a method for manufacturing a finger grip according to the present invention, the grip pad is formed by applying the grip pad injection molding material directly onto at least one the distal facing surfaces of the finger grip body, wherein the grip pad is particular made in one piece, preferably with just one injection point, which is in particular located in an outer third of the grip pad in radial or transversal direction.

The fifth aspect of the present invention relates to a syringe assembly for administering a liquid composition to a human or animal body, that means to any subject, comprising at least a longitudinal syringe barrel having an inner lumen for receiving and storing a liquid composition to be administered, a finger grip coupled to a proximal end of said barrel and a plunger having a plunger rod and a plunger head, wherein the plunger rod is disposed at least partly within the inner lumen of the barrel's distal end. A syringe assembly according to the fifth aspect of the present invention is characterized in that the plunger is a plunger according to the present invention or a plunger made by a method according to the present invention, and/or in that the finger grip is a finger grip according to the present invention and/or a finger grip made by a method according to the present invention.

The syringe assembly in general may be an assembly for administering a one-component liquid composition or a two-component or a multi-component liquid composition. However, in a preferred embodiment, the syringe assembly is configured for administering a one-component ready-to-use mixed composition.

In one embodiment of the present invention the syringe barrel of the syringe assembly is made of plastic, in particular of cycloolefin copolymer (COC). Using a syringe barrel made of plastic reduces the risk of needle pop-off during administering, in particular during ejection or injection of high viscous fluids as dermal filler are, since with a plastic barrel a better syringe-needle hub connection can be established compared to a barrel made of glass.

Further, using plastic for the syringe barrel allows providing a syringe barrel having a Luer lock connector integrally manufactured with said barrel. Thereby, the risk of needle pop-off and/or disconnection between a needle assembly or a cannula and the syringe barrel can be reduced. Further, a plastic syringe barrel can easily be manufactured by injection molding, which allows manufacturing the syringe barrel with less tolerances resulting in more reliable needle-syringe connections and less extrusion forces during injection of the content stored in the inner lumen of said syringe barrel. In addition, a better geometric accuracy of the syringe barrel can be achieved allowing a more precise and stronger and more secure coupling of the finger grip and the plunger to said syringe barrel. Moreover, the risk of failure of the syringe barrel flange of a plastic syringe can be reduced compared to a glass syringe.

The inner surface of the syringe barrel may be siliconized. Thereby, extrusion forces can be reduced.

In one embodiment of the present invention the syringe barrel of the syringe assembly according to the present may have an inner lumen for receiving and storing a volume of 2 ml or less of a composition to be administered, wherein the syringe barrel may in particular be configured for receiving and storing an amount of 1 ml or less, for example of 0.5 ml, of a composition, in particular of a liquid composition of a dermal filler, as for example of a hyaluronic acid dermal filler.

The inner diameter of the inner volume of the syringe barrel therefor may for example be smaller than of a standard syringe barrel according to standard ISO 11040-6 and preferably longer, wherein a diameter/length ratio is preferably about 5/70. The longer an inner diameter of the syringe barrel has the advantage that the extrusion force is lowered compared to a syringe barrel according to standard ISO 11040-6. This results an easier, more controlled and safer use. Also, the extruded volume can be better controlled is less product is excluded per millimeter of plunger movement in longitudinal direction during ejection.

With a syringe assembly according to the present invention, a very easy, comfortable and safe to use syringe assembly for administering a liquid composition, in particular for injection of dermal filler, in particular for hyaluronic acid dermal filler, can be provided, wherein the syringe assembly respectively the single parts of said syringe assembly are easy to manufacture, enable a good storage of a liquid composition to be administered and an easy handling by a practitioner.

Siliconization of the inner surface of the barrel has the advantage that a good lubricant layer can be applied on the inner surface of the barrel by keeping the free silicon to a minimum preventing interactions with a dermal filler. Hence, by siliconization of the inner barrel surface lower extrusion forces can be achieved what results also in an easier, more controlled and safer use. Lower extrusion forces further help to reduce the risk of mechanical failure.

A grip pad and/or symbols integrated in the grip pad improve usability and safety due to the easy optical recognition. By using a TPE grip pad, a good finger contact can be achieved which enables controlled and safer administering. By a colored grip pad, optical recognition can be improved further.

In one embodiment of a syringe assembly according to the present invention, the syringe barrel comprises a Luer lock connector at its distal end, which is in particular integrally formed with the syringe barrel. Further, the syringe assembly may in particular include a tip cap covering and sealing the distal end of the syringe barrel. A Luer-lock connector has compared to the Luer-cone connector the advantage that a safer and locked connection, which is also releasable, can be established.

Luer connections and connectors are generally known in prior art and standardised, wherein Luer connections generally comprise a male Luer connector and a corresponding female Luer connector. Luer-lock connections are for example described in DIN EN ISO 80369-7:2017-10. They are characterized by a special 6% cone, wherein a Luer-lock connection also comprises a threaded connection.

The tip cap may be a two-part tip cap comprising a rigid part and an elastomeric part, wherein the maximum outer diameter of the rigid part is preferably less or equal to the outer diameter of the Luer lock connector of the syringe barrel. Thereby, assembly, storage and unpacking and usage of a prefilled syringe assembly can be made safer, since thereby the risk of snagging and unintended removing or damage of the tip cap can be reduced.

In one embodiment the rigid part may comprise an outer thread and may be screwed to and engaged with the inner thread of the Luer lock connector of the syringe barrel, wherein the elastomeric part of the tip cap may be inserted into the rigid part and attached to the Luer cone of the Luer lock connector, thereby closing an outlet channel of the syringe barrel.

The sixth aspect of the present invention relates to a kit comprising parts of the syringe assembly according to the present invention configured to be assembled to a syringe assembly according to the invention and as pre-described, wherein the kit comprises at least a longitudinal syringe barrel having an inner lumen for receiving and storing a liquid composition to be administered, a finger grip configured to be coupled to the proximal end of said barrel, and a plunger having a plunger rod and a plunger head, the plunger rod configured to be disposed at least partly within the inner lumen of the barrel with its distal end. A kit according to the present invention is characterized in that the plunger is a plunger according to the present invention or a plunger made by a method according to the present invention and/or that the finger grip is a finger grip according to the present invention or a finger grip made by a method according to the present invention, wherein in particular the plunger rod is configured to be inserted through the center bore of the finger grip body.

With a kit according to the present invention a very easy, comfortable and safe to use kit comprising parts of a syringe assembly for administering of a liquid composition, in particular for injection of dermal filler, in particular for hyaluronic acid dermal fill, can be provided, wherein the kit respectively the single parts of said kit are easy to manufacture, enable good storage of a liquid composition to be administered and an easy handling by practitioner.

In one embodiment of a kit according to the present invention and/or of a syringe assembly according to the present invention, in particular in a preferred embodiment, the syringe barrel is prefilled, in particular with a liquid composition, in particular with a soft tissue filler material, for example with one of the following soft tissue filler materials:
hyaluronic acid (hyaluronan) (HA),
carboxymethylcellulose (CMC),
calcium hydroxyl apatite in suspension with a gel carrier such as CMC or HA collagen,
another natural polysaccharide (alginate, heparosan, chitosan etc.),
a synthetic filler material (e.g., polyethylene oxide, poly-L-lactic acid (PLLA), poly(methyl)methacrylate (PMA), polyacrylamide (PA), polyvinyl alcohol (PVA)) or
liquid injectable silicone.

Generally, the filler material like hyaluronic acid and the like may be crosslinked and/or uncrosslinked. Furthermore, the soft tissue filler may contain additional substances like vitamins, antioxidants, local anesthetics (e.g. lidocaine), hydroxyapatite particles etc.

In particular, the syringe barrel may be prefilled with a filler material based on crosslinked hyaluronic acid (HA), in particular 1,4-butanediol diglycidyl ether (BDDE)-crosslinked hyaluronic acid (HA) dermal filler.

The intended use is not particularly limited. However, the most important use is the use for cosmetic purposes, for example for the treatment of skin lines.

That means, generally, a syringe assembly or a kit according to the present invention may be provided with an empty syringe barrel or with a prefilled syringe barrel. However, in a preferred embodiment, a syringe assembly or a kit according to the present invention is provided with a prefilled syringe barrel, in particular prefilled with one of the above-mentioned soft tissue filler materials.

As used herein, the term “soft tissue filler” broadly refers to a material designed to add volume to areas of soft tissue deficiency. A “dermal filler” or “dermal filler composition”, as used herein, is generally a substance that adds, replaces or augments volume under the skin leading to, e.g., smoothened skin wrinkles, augmented lips, improved skin appearance, or treated scars. A dermal filler composition of the present invention is, like a soft tissue filler, generally “injectable”. Further, a dermal filler is generally used in the dermis area, such as below the epidermis or above the hypodermis and as such may be injected subcutaneously or intradermally, or some combinations.

Optionally, the kit can comprise instructions for use. The “instructions for use” may, for example, be instructions for use in cosmetic applications and/or for injecting a soft tissue filler (e.g. a dermal filler) into the human body, including replacing or filling of a biological tissue or increasing the volume of a biological tissue for the purpose of cosmetic applications. In particular, the kit may comprise instructions for use as a soft tissue filler or a dermal filler in cosmetic treatments.

The seventh aspect of the present invention relates to the use of a syringe assembly or a kit according to the present invention, in particular for cosmetic or therapeutic application, particularly for replacing or filling a biological tissue or increasing volume of a biological tissue, wherein an effective amount of a liquid composition, particularly an effective amount of a dermal filler composition, is to be administered or is administered to a subject by using a syringe assemble according to the present invention and/or a kit according to the present invention.

The dermal filler composition may in general be administered in an effective amount to a subject by injection, such as, e.g., by subcutaneous or intradermal injection. For example, the composition may be intradermally or subcutaneously injected using the serial puncture technique, preferably with an injection angle of minimum 1, 2, 3, 5, 8, 10, 12, 15, 20, 25, 30, 35, 40 or 45 degrees up to an injection angle of maximum 12, 15, 20, 25, 30, 35, 40, 45, 60, 75 or 90 degrees, wherein each of the aforementioned minimum values can be combined with any one of the previously mentioned maximum injection angles. That means, an injection angle may be, for example, in a range from 8 to 25 degrees or in a range from 12 to 60 degrees or in a range from 5 to 15 degrees and so on. The term “injection angle”, as used herein, refers to the angle between said first longitudinal axis of an injection needle and the line projected by the needle onto the skin surface.

By choosing an appropriate injection angle, injection results may be improved. In many cases, small injection angles, for example in a range from 2, 5 or 8 to 12 or 15 degrees, are very advantageous. In other cases, larger injection angles may be better, for example in a range from 25, 30 or 45 to 60 or 90 degrees.

The term “effective amount” refers to the amount of the soft tissue filler composition sufficient to effect beneficial or desired cosmetic (aesthetic) results.

A “subject”, as used herein, may be any individual or patient, e.g., a mammal and, preferably, a human, in need of a treatment or application for cosmetic (aesthetic) purposes.

The present invention described above will now be further explained by the following, non-limiting embodiments and examples, wherein further optional features of the inventions are disclosed in the drawings and in the description of these drawings. All features and all combinations of features described above and/or outlined below and/or only illustrated in the drawings can be realized in an embodiment of the invention in the combination described or stand alone or in at least one other combination not described explicitly herein, as this combination is technically reasonable.

### BRIEF DESCRIPTION OF THE DRAWINGS

The accompanying drawings, which are incorporated herein and form a part of the specification, illustrate schematically preferred embodiments of the inventions described above, in particular of the present invention, and together with the description, serve to explain the principles and at least partly the features of the described inventions, wherein for same parts same reference signs are used. It is illustrated in:

FIG. 1 a perspective view of a first example of an embodiment of a syringe assembly according to the present invention comprising a first example of a plunger according to the present invention and an example of a finger grip according to the present invention,

FIG. 2 a perspective view of the second example of an embodiment of the syringe assembly according to the present invention comprising a second example of a plunger according to the present invention and the same finger grip as the syringe assembly in FIG. 1,

FIG. 3 a side view of the syringe assembly of FIG. 1,

FIG. 4 a longitudinal section through the syringe assembly of FIGS. 1 and 3,

FIG. 5 a top view on the proximal facing surface of the plunger of the syringe assembly of FIG. 1,

FIG. 6 a top view on the proximal facing surface of the plunger of the syringe assembly of FIG. 2,

FIG. 7 a first side view of the plunger of FIG. 1,

FIG. 8 a first side view of the plunger of FIG. 1,

FIG. 9 a longitudinal section through the plunger of FIG. 1,

FIG. 10 an enlarged illustration of a cross section of the plunger of FIG. 10 along A-A′,

FIG. 11 an enlarged illustration of the distal end of the plunger shown in FIG. 9,

FIG. 12 an enlarged illustration of the proximal end of the plunger shown in FIG. 9,

FIG. 13 an enlarged illustration of a bottom view of the piston of the syringe assembly of FIG. 4,

FIG. 14 an enlarged illustration of a cross section of the piston of the syringe assembly of FIG. 4,

FIG. 15 a first perspective view of the finger grip according to the present invention of the syringe assembly illustrated in some of the previous Figs.,

FIG. 16 a second perspective view of the finger grip illustrated in FIG. 15,

FIG. 17 a top view of the finger grip illustrated in FIGS. 15 and 16,

FIG. 18 a side view of the finger grip illustrated in FIGS. 15 to 17,

FIG. 19 a perspective view of the syringe barrel of the syringe assembly illustrated in FIG. 1,

FIG. 20 a longitudinal section through the syringe barrel of FIG. 19, and

FIG. 21 an enlarged illustration of the tip cap at the distal end of the syringe barrel illustrated in FIG. 20.

### DETAILED DESCRIPTION OF SEVERAL EMBODIMENTS OF THE INVENTION

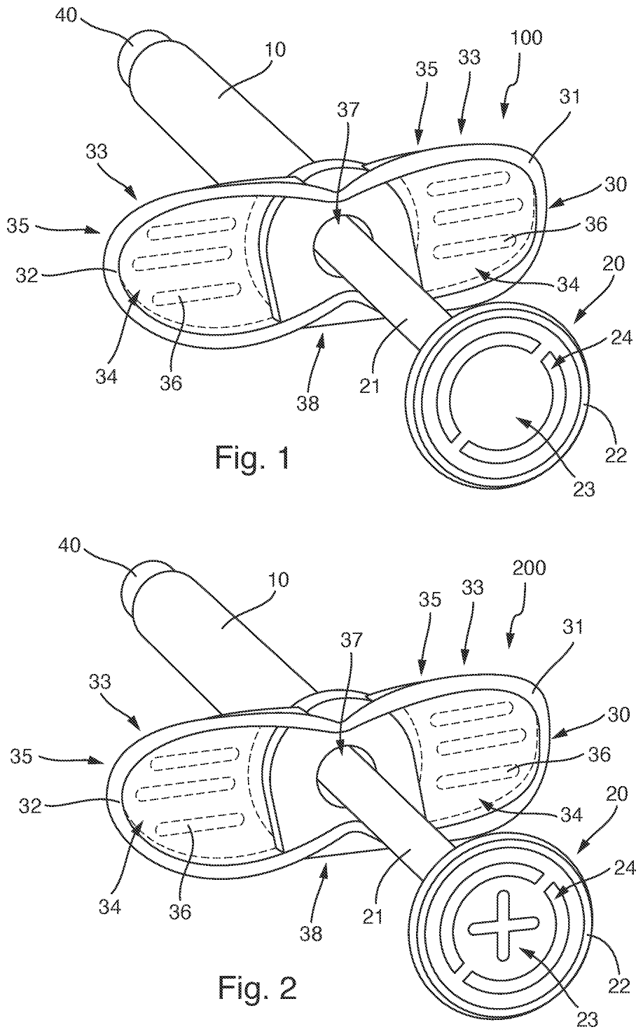

In FIG. 1 a perspective view of a first example of an embodiment of a syringe assembly 100 according to the present invention is illustrated, wherein this syringe assembly 100 comprises a first example of a plunger 20 according to the present invention and an example of a finger grip 30 according to the present invention, wherein the syringe assembly 100 is assembled by parts of an example of a kit according to the present invention, and wherein the plunger 20 respectively the finger 30 each are made by a method according to the present invention.

The syringe assembly 100 further comprises a syringe barrel 10 having an inner lumen 13 (see e.g. FIG. 4) being prefilled with a liquid composition (not shown), wherein the liquid composition is a hyaluronic acid dermal filler. The proximal end of the syringe barrel 10 is closed and sealed by a tip cap 40.

The plunger 20 can be moved into the syringe barrel 10 along a longitudinal axis A of said syringe barrel 10 for dispensing the composition stored in the inner lumen 13 (see e.g. FIG. 4) of said syringe barrel 10, in particular for ejection of that composition out of the barrel 10 after removing the tip cap 40.

The plunger 20 comprises a plunger head 22 and a plunger rod 21 each made by injection molding of polypropylene PP, wherein the plunger head 22 and the plunger rod 21 are made in one piece. That means, they are integrally molded. The plunger head 22 comprises a proximal facing surface 24 onto which also by injection molding a grip pad 23 has been applied for facilitating gripping respectively for improving a contact of a user's thumb. The grip pad 23 is made of thermoplastic elastomere (TPE) which allows a safe and precise support of the thumb of a user during use of said syringe assembly 100 and therewith a precise administering of the composition stored in the syringe barrel 10.

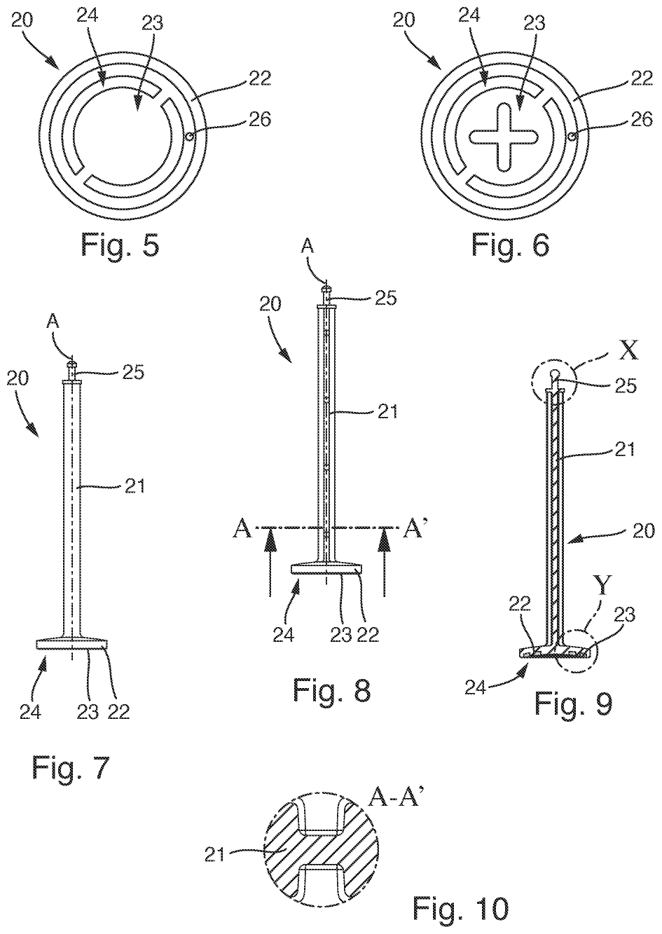

In this example of a plunger 20 according to the present invention the grip pad 23 attached to the plunger head 22 is made in one piece via one injection point 26 only (see FIG. 5). The grip pad 23 extends almost over the whole proximal facing surface 24 of the plunger head 22 except of some areas not being filled or covered with grip pad material 23. In this example of a plunger 20 according to the present invention the areas not being filled or covered with grip pad material are completely surrounded by grip pad material 23.

Due to the fact that compositions as the hyaluronic acid dermal filler being stored in the syringe barrel 10 of syringe assembly 100 require high extrusion forces respectively high ejection forces because of their high viscosity, plunger rod 21 has to be resistive against kinking. To avoid mechanical failure of the plunger rod 21 during use, the plunger rod 21 has a double-mushroom shaped profile almost over its whole length, see also FIGS. 4 and 7 to 10. Thereby, a very good mechanical strength can be achieved, in particular also for plungers 20 configured to be used with syringe barrels 10 for syringe assemblies 100 and 200 as illustrated in FIGS. 1 and 2, which are configured for receiving and storing an amount of 1 ml of the composition to be administered, wherein the inner lumen 13 of the syringe barrel 10 has a relatively small inner diameter, in particular a smaller inner diameter and a greater length compared to an inner lumen of a standard syringe barrel according to standard ISO11040-6. By the reduced inner diameter of the inner lumen 13, i.e. by its smaller inner diameter, the extrusion forces during use can be reduced, what also helps to reduce the risk of mechanical failure of the plunger 20, in particular of the plunger rod 21. In an alternative embodiment, plunger rod 21 may comprise a massive cross-section in particular an oval or a circular or a square cross-section, wherein said massive, in particular oval or circular or square cross-section, may extend only over one section of the length of the plunger rod 21 or over several sections in longitudinal direction of the plunger rod 21 or may extend over the complete length of the plunger rod 21. Size and geometry of the cross-section of the plunger rod 21 may be constant over the length of plunger rod 21 or vary. In one embodiment, the geometry and/or size of the cross-section area of the plunger rod may, for example, be constant over at least 80% or 90% of the complete length of the plunger rod.

The finger grip 30 comprises a finger grip body, also made by injection molding of polypropylene PP in one piece, having two flanges or wings 31 and 32 extending radially in opposite directions from a center bore 37 in the finger grip body. Through the center bore 37 the plunger 20 is inserted. The wings 31, 32 are extending to the right respectively to the left of the center bore 37. Each wing 31, 32 serves as a grip portion and has a finger contact area 35 onto which at least one finger of a user can be put partly during administering of the composition stored in the syringe barrel 10 to support the finger whilst administering, wherein each wing 31, 32 comprises a distal facing surface 33 and proximal facing surface 34.

According to the present invention, the finger grip 30 comprises at least one grip pad 35, in this example two grip pads 35, wherein each grip pad is attached to the distal facing surface 33 of one of the wings 31, 32 and extends almost over the whole distal facing surface 33. Both grip pads 35 are made by injection molding, wherein the grip pads 35 in this example of a finger grip 30 according to the present invention have been applied directly onto the distal facing surfaces 33 when injection molded.

The grip pads 35 attached to the flanges 31 and 32 also made of a thermoplastic elastomer (TPE) in one piece each having one injection point 26 (see FIGS. 16 and 17) only. These grip pads 35 also improve grasping of the syringe assembly 100 and help prevent slipping of the user's fingers during administering of the composition stored in the syringe barrel 10.

The grip pads 35 of the finger grip 30 also comprise areas 36 not being filled with grip pad injection molding material 35, wherein these areas are forming parallel and transversally extending stripes 36 completely being surrounded by grip pad material 35.

The finger grip 30 is coupled to the proximal end of the syringe barrel 10 by a flange 12 (see e.g. FIG. 4) formed at the proximal end of the syringe barrel 10, wherein flange 12 is inserted into a side slot 38 in the finger grip 30 (see e.g. FIG. 4). Through the center bore 37 the plunger rod 21 of the plunger 20 is inserted such that the plunger rod 21 is disposed with its distal end at least partly in the inner lumen 13 of the syringe barrel 10.

For easy distinguishing and avoiding mis-administering of the composition stored in the syringe barrel 10 of the syringe assembly 100, the grip pad 23 attached to the plunger head 22 and the grip pads 35 attached to the finger grip body 30 respectively to its wings 31 and 32 are made of colored grip pad material, in particular of colored TPE, wherein the color is associated to that particular composition of a hyaluronic acid dermal filler stored in the syringe barrel 10. This allows a medical practitioner to recognize at the first glance which composition the respective syringe assembly 100 intended to be used contains.

In FIG. 2 a second example of an alternative embodiment of a syringe assembly 200 according to the present invention is shown, wherein in this embodiment 200 the grip pad 23 attached to the plunger head 22, in particular its proximal facing surface 24, is designed in such a way that it represents a symbol, in particular a “+”. In this case, that symbol “+” is associated to a composition containing the active ingredient Lidocaine. This design of the grip pad 23 of the plunger 20 allows a medical practitioner to recognize at a first glance, that a syringe assembly 200 being filled with a composition containing Lidocaine has been chosen. Thereby, the risk of mis-administering caused by mixing up syringe assemblies containing different compositions to be administered can be reduced.

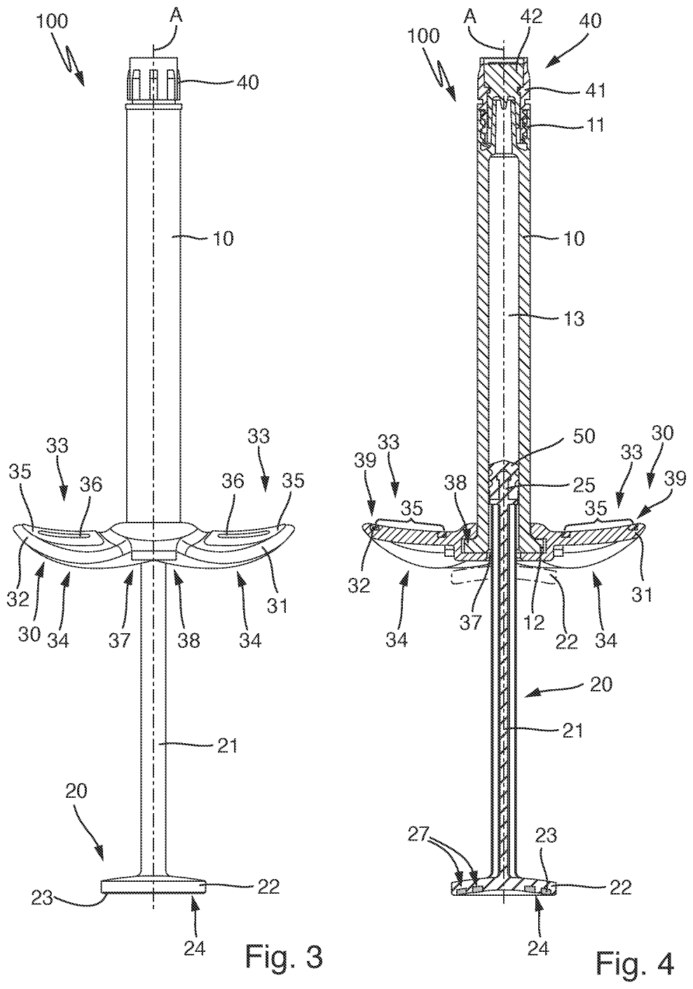

FIG. 3 shows a side view of the syringe assembly 100 illustrated in FIG. 2, wherein in this illustration the arrangement of the grip pads 35 on the finger grip 30, in particular its distal facing surfaces 33 is visible clearly.

FIG. 4 shows a longitudinal section through the syringe assembly 100 of FIGS. 1 and 3, wherein in this illustration the coupling of the plunger 20, in particular of the plunger rod 21, with the syringe barrel 10 and the finger grip 30 and as well the coupling between the finger grip 30 and the syringe barrel 10 can be seen.

In particular from the illustration in FIG. 4 can be seen, how flange 12 at the proximal end of the syringe barrel 10 is inserted into the slot 38 in the finger grip 30 for coupling of the syringe barrel 10 to the finger grip body 30. Further it can be seen that the distal end of the plunger rod 21 is inserted through the center bore 37 into the inner lumen of the syringe barrel 10. At its distal end 25 the plunger 20 is coupled to a piston 50, in particular by a snap-fit connection, wherein the piston 50 in this example of a syringe assembly 100 is made of FM257 what an elastomeric material is.

To achieve good adhesion between the grip pad 23 and the proximal facing surface 24 of the plunger head 22 and between the grip pads 35 and the distal facing surfaces 33 of the finger grip body 30, the surfaces to which the grip pads 23 respectively 35 are applied comprises several grooves 27, 39 each (see FIGS. 4 and 12), which are filled by grip pad injection molding material during injection molding of the grip pads 23, 35.

FIG. 4 further shows the closing and sealing of the distal end of the syringe barrel 10 by the tip cap 40 in detail. For coupling said tip cap 40 to the syringe barrel 10 the syringe barrel 10 comprises a Luer lock connector 11 at its distal end. The tip cap 40 comprises a rigid outer part 41 and an elastomeric inner part 42. The rigid outer part 41 is threadedly engaged with the inner threads of the Luer lock connector 11. The elastomeric inner part 42 of the tip cap 40 is inserted into the rigid outer part 41 extending partly with a frustoconical protrusion into an outlet channel 15 of the distal end of the syringe barrel 10, see also FIGS. 20 and 21.

FIG. 5 shows a top view of the plunger 20 of the syringe assembly 100 illustrated in FIG. 1, wherein in FIG. 5 the one and only injection point 26 used for manufacturing of the in one piece made grip pad 23 is indicated. In this example, the injection point 26 is located near the outer edge of the grip pad 23. For improved adhesion of the grip pad injection molding material 23 to the proximal facing surface 24 of the plunger head 22 of plunger 20, the plunger 20 is made by an injection tool (not shown) having a tool surface, which is responsible for shaping of the proximal facing surface 24, which has been eroded resulting in a surface roughness according to VDI 3400 Ref. 27 with Ra=2.24 μm and Rmax=9.0 μm.

FIG. 6 shows a top view of the plunger 20 of the syringe assembly 200 is illustrated in FIG. 2, wherein the grip pad 23 attached to the proximal facing surface 24 of the plunger head 22 of this plunger 20 is also made in one piece by injection molding by using one injection point 26 only. The injection point 26 is also located near the outer edge respectively the outer circumference of grip pad 23. In difference to the grip pad 23 of the plunger 20 illustrated in FIG. 5, the grip pad 23 of the plunger illustrated in FIG. 6 is designed in such a way that it represents a symbol, in particular a “+”, as already described in relation to FIG. 2, by which is indicated that the composition the syringe barrel 10 of the syringe assembly 200 is prefilled with, contains Lidocaine.

FIGS. 7 and 8 show different side views of the plunger 20 of the syringe assembly 100 as illustrated in FIG. 1, wherein the illustration in FIG. 8 shows the plunger 20 rotated about 90° around its longitudinal axis A compared to the illustration in FIG. 7.

FIG. 9 shows an illustration of a longitudinal section of the plunger 20 in accordance with FIGS. 1, 7 and 8, wherein in FIG. 9 the double-mushroom-shaped profile of the plunger rod 21 is visible.

FIG. 10 shows an enlarged illustration of a cross section of the plunger of FIG. 10 along section plane A-A′ by which the double profile of the plunger rod 21 becomes apparent.

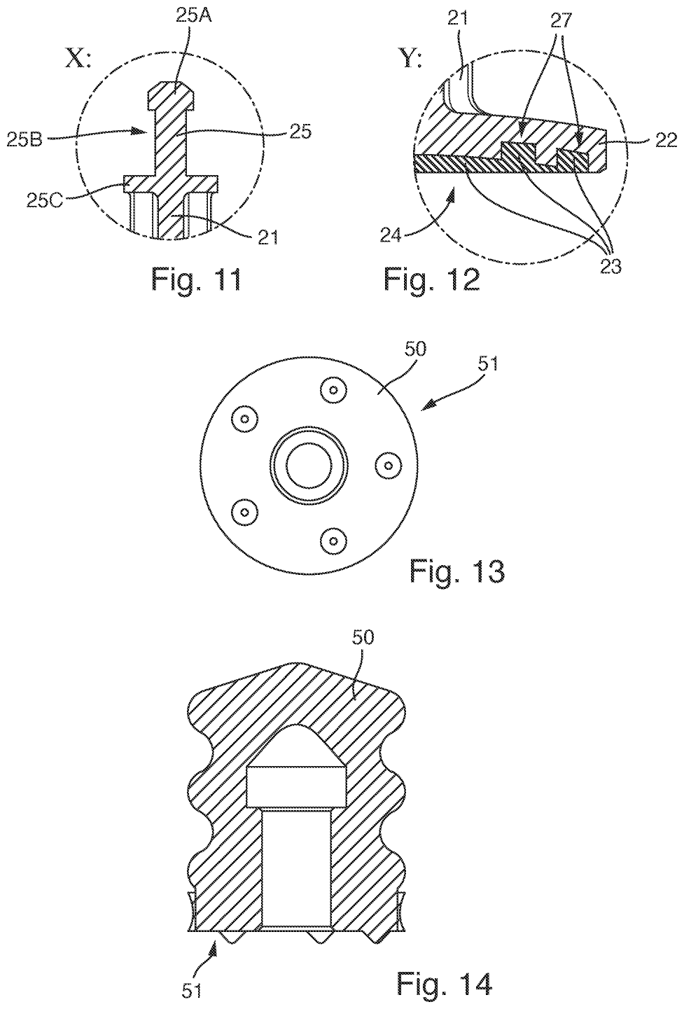

FIG. 11 shows an enlarged illustration of the distal end 25 of the previously described plunger 20 illustrated in FIG. 9, wherein the plunger rod 21 can be coupled with its distal end 25 to a piston by a snap fit connection (see e.g. FIGS. 4 and 14—piston 50). The distal end 25 of the plunger rod 21 comprises a mushroom-shaped tip 25A. In this example, the plunger tip 25 further comprises a piston receiving section 25B arranged adjacent to the mushroom-shaped tip 25A a in proximal direction having a smaller diameter than the mushroom-shaped tip 25A. The piston receiving section 25B is limited in proximal direction by a plate disc-shaped axial piston stop 25C for axial fixation of a piston (not shown apparently here). Thereby, a secure connection between the distal end 25 of the plunger rod 21 and a piston 50 can be achieved.

FIG. 12 shows an enlarged illustration of the proximal end of the plunger 20 shown in FIG. 9. The proximal facing surface 24 of plunger head 22 comprises two circular grooves 27 arranged concentric around the longitudinal axis A, wherein the grooves have been filled by grip pad injection molding material 23 during applying the grip pad material 23 onto the proximal facing surface 24 of the plunger head 22. By these grooves 27 the contact area between the proximal facing surface 24 and the grip pad material 23 can be increased, resulting in better adhesion between the grip pad material 23 and the plunger head 22.

FIG. 13 shows an enlarged illustration of a bottom view of the piston 50 of the syringe assembly 100 illustrated in FIG. 4 and FIG. 14 an enlarged illustration of a cross section of said piston 50, wherein said piston 50 is configured to be snap-fit connected with the mushroom-shaped tip 25 of plunger rod 21. For a tight fit with the walls of the inner lumen 13 of the syringe barrel 10 said piston 50 comprises a wave-shaped outer geometry. On its bottom side 51, the piston 50 comprises several protrusions not indicated configured to get in contact with the axial piston stop 25C of the plunger rod 21.

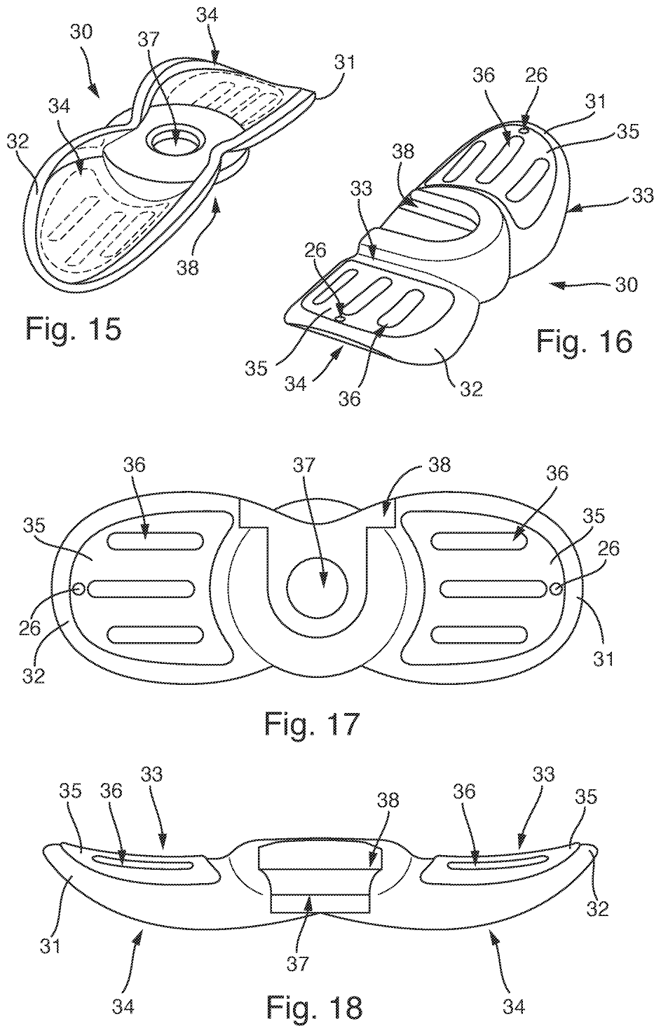

FIGS. 15 to 18 show different views of the example of the finger grip 30 according to the present invention of the syringe assemblies 100 and 200 as illustrated in FIGS. 1 and 2, wherein from FIGS. 15 to 18 in particular the curved shape of the wings 31 and 32, the design and arrangement of the grip pads 35 on the distal facing surfaces 33, the center bore 37 and the side slot 38 for coupling of the finger grip 30 to the proximal end of the syringe barrel 10 become apparent.

Like the plunger head grip pads 23 of the plunger 20 of the syringe assemblies 100 and 200 illustrated in FIGS. 1 and 2, also the grip pads 35 of the finger grip 30 are made by injection molding. The grip pads 35 are also made in one piece of colored TPE by using one injection point 26 only, wherein the grip pad material 35 has been applied to the distal facing surfaces 33 of the wings 31, 32 directly. The injection points 26 are also located at the outer edge of the grip pads 35, which also extend almost completely over the corresponding surface 33. As described above, some areas 36 forming parallel and transversal extending stripes 36 are not being filled or covered with grip pad material 35, but are completely surrounded by the grip pad material 35 to achieve a good economic haptic and a good finger contact with a reduced risk of slipping of any finger of a user.

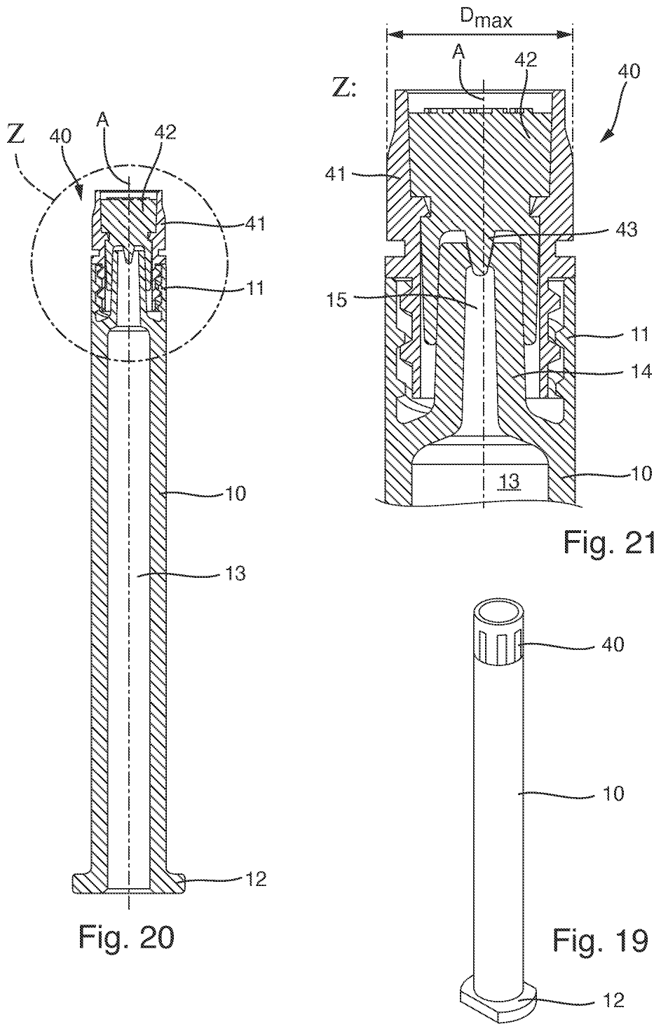

FIG. 19 shows a perspective individual view of the syringe barrel 10 of the syringe assembly 100 as illustrated in FIG. 1, wherein the inner lumen 13 of said barrel 10 is prefilled with a hyaluronic acid dermal filler. The distal end of said barrel 10 is closed and sealed by the tip cap 40. The proximal end of the syringe barrel 10 comprises a flange 12, which can be inserted laterally into the slot 38 of the finger grip 30 for coupling of said syringe barrel 10 to the finger grip 30 respectively the finger grip 30 to said barrel 10.

FIG. 20 shows a longitudinal section through the barrel 10 of FIG. 19.

FIG. 21 shows an enlarged illustration of the tip cap 40 of FIG. 20. As can be seen, the syringe barrel 10 comprises a Luer lock connector 11 at its distal end, wherein the Luer connector comprises inner threads and a Luer cone 14 surrounding an outlet channel 15, which is in fluid communication with the inner lumen 13 of the syringe barrel 10.

The distal end of the syringe barrel 10 is closed and sealed by the tip cap 40, which comprises an outer rigid part 41, engaged with the inner threads of the Luer lock connector 11, and an inner elastomeric part 42, which is inserted into the outer rigid part 41 thereby closing and sealing the outlet channel 15 of the syringe barrel 10.

In this example, the maximum outer diameter Dmax of the rigid part 41 is less or equal to the outer diameter of the Luer connector 11 of the syringe barrel 11. Thereby a risk of unintended snagging or damage of the tip cap 40 and therewith of the closing and sealing of the prefilled syringe barrel 10 can be reduced. In particular, the outer wall of the rigid part is at least partly in flush with the outer wall of the Luer lock connector.

All of the features described herein with respect to one aspect of the present invention may also be realized alone or in combination in any other of the subjects according to any other aspect of the present invention, so far this is technically possible.

### LIST OF REFERENCE SIGNS

100 first example of an embodiment of a syringe assembly according to the present invention
200 second example of an embodiment of a syringe assembly according to the present invention
10 syringe barrel
11 Luer lock connector
12 flange of the syringe barrel at the proximal end of the syringe barrel
13 Luer cone of the Luer lock connector
15 outlet channel
20 example of an embodiment of a plunger according to the present invention
21 plunger rod
22 plunger head
23 grip pad of the plunger head
24 proximal facing surface of the plunger head
25 plunger tip at the distal end of the plunger rod
25A mushroom-shaped tip of the plunger rod
25B piston receiving portion
25C axial piston stop
26 injection point
27 groove in the proximal facing surface of the plunger head
30 example of an embodiment of the finger grip according to the present invention
31 right flange or wing of the finger grip body
32 left flange or wing of the finger grip body
33 distal facing surface of the finger grip body
34 proximal facing surface of the finger grip body
35 grip pad of the finger grip
36 area not being filled or covered by grip pad injection molding material of the
finger grip designed as a stripe
37 center bore in the finger grip body
38 slot for finally insertion of a proximal flange of a syringe barrel
39 groove in the distal facing surface of the finger grip body
40 tip cap
41 rigid part of the tip cap (outer part)
42 elastomeric part of the tip cap (inner part)
50 piston
51 bottom side of the piston
A longitudinal axis of the syringe barrel
Dmax maximum outer diameter of the rigid part of the tip cap

1. A plunger for a syringe assembly for administering a liquid composition,
wherein the plunger comprises a plunger rod and a plunger head,
wherein the plunger rod comprises a proximal end and a distal end, and
wherein the plunger head is attached to the proximal end of the plunger rod and comprises a proximal facing surface configured to serve as a thumb plate during administering,
wherein the plunger comprises a grip pad attached to the proximal facing surface of the plunger head and extending at least partly over said proximal facing surface, wherein the grip pad is made by injection molding.

2. The plunger according to claim 1, wherein the grip pad on the plunger head is made of a thermoplastic elastomere (TPE), optionally a coloured thermoplastic elastomere (cTPE).

3. The plunger according to claim 1, wherein the proximal facing surface of the plunger head comprises at least one deepening, optionally at least one groove, which is filled by grip pad injection molding material.

4. The plunger according to claim 1, wherein the grip pad on the plunger head represents a symbol (“+”), optionally a letter or another character selected from the ASCII-Code.

5. The plunger according to claim 1, wherein the grip pad on the plunger head is made in one piece comprising one injection point only.

6. The plunger according to claim 1, wherein the plunger rod comprises at least one section in a longitudinal direction with an at least substantially double-T-profile shape, a double-T-profile-like shape, a I-profile shape or a I-profile-like shape.

7. A method for manufacturing a plunger according to claim 1, comprising at least:
manufacturing the plunger head,
forming the grip pad by injection molding.

8. A finger grip for a syringe assembly for administering a liquid composition, comprising a finger grip body with two flanges extending radially in opposite directions from a center bore in the finger grip body through which a plunger of a syringe assembly can be inserted, each flange serving as a grip portion and having a finger contact area onto which at least one finger of a user can be put partly during administering to support the finger whilst administering, wherein each flange comprises a distal facing surface and a proximal facing surface,
wherein the finger grip comprises a grip pad attached to the distal facing surface of at least one of the flanges extending at least partly over said distal facing surface, wherein the grip pad is made by injection molding.

9. The finger grip according to claim 8, wherein at least one grip pad on the finger grip is made of a thermoplastic elastomere (TPE), optionally a coloured thermoplastic elastomere (cTPE).

10. The finger grip according to claim 8, wherein at least one grip pad on one of the finger grip flanges is made in one piece comprising one injection point only.

11. A method for manufacturing a finger grip according to claim 8, comprising at least:
manufacturing a finger grip body including two flanges extending radially in opposite directions from a center bore (37) in the finger grip body (30) through which a plunger of a syringe assembly can be inserted, each flange serving as a grip portion and having a finger contact area onto which at least one finger of a user can be put partly during administering to support the finger whilst administering, wherein each flange comprises a distal facing surface and a proximal facing surface, and
forming the grip pad by injection molding.

12. A syringe assembly for administering a liquid composition to a human or animal body, comprising at least:
a longitudinal syringe barrel having an inner lumen for receiving and storing a liquid composition to be administered,
a finger grip coupled to a proximal end of said barrel, and
a plunger having a plunger rod and a plunger head, the plunger rod being disposed at least partly within the inner lumen of the barrel with a distal end thereof,
wherein the plunger is a plunger according to claim 1 and/or the finger grip is a finger grip comprising a finger grip body through which the plunger of a syringe assembly can be inserted.

13. The syringe assembly according to claim 12, wherein the syringe barrel comprises a Luer lock connector at a distal end thereof integrally formed with the syringe barrel, wherein the syringe assembly further comprises a tip cap, the tip cap covering and sealing the distal end of the syringe barrel, wherein the tip cap is a two-part tip cap comprising a rigid part and an elastomeric part, wherein the maximum outer diameter (Dmax) of the rigid part is less or equal to the outer diameter of the Luer lock connector of the syringe barrel.

14. A kit comprising parts of a syringe assembly according to claim 12, configured to be assembled to a syringe assembly according to claim 12, wherein the kit comprises at least:
a longitudinal syringe barrel having an inner lumen for receiving and storing a liquid composition to be administered,
a finger grip configured to be coupled to a proximal end of said barrel, and
a plunger having a plunger rod and a plunger head, wherein the plunger rod is configured to be disposed at least partly within the inner lumen of said barrel with thereof a distal end thereof,
wherein the plunger is a plunger according to claim 1 and/or the finger grip is a finger grip comprising a finger grip body through which the plunger of a syringe assembly can be inserted.

15. A product comprising a syringe assembly according to claim 12 or a kit thereof for cosmetic or therapeutic application, optionally for replacing or filling a biological tissue or increasing volume of a biological tissue, wherein said product comprises an effective amount of a liquid composition, optionally an effective amount of a dermal filler composition, which is adapted to be administered to a subject.
